SEQUENCE LISTING (17 sequences)

SEQ ID NO: 1 - protein, 15 aa, Artificial Sequence
  Linker
GGGGSGGGGSGGGGS

SEQ ID NO: 2 - protein, 15 aa, Artificial Sequence
  Linker
SGGGSGGGGSGGGGS

SEQ ID NO: 3 - protein, 18 aa, Artificial Sequence
  Linker
GSTSGSGKPGSGEGSTKG

SEQ ID NO: 4 - protein, 26 aa, Artificial Sequence
  Linker
SSADDAKKDAAKKDDAKKDDAKKDAS

SEQ ID NO: 5 - protein, 16 aa, Artificial Sequence
  Linker
GGGGSGGGGSCGGGGS

SEQ ID NO: 6 - protein, 14 aa, Artificial Sequence
  Linker
EGKSSGSGSESKVD

SEQ ID NO: 7 - protein, 16 aa, Artificial Sequence
  Linker
DGGGSGGGGSGGGGSS

SEQ ID NO: 8 - protein, 106 aa, Artificial Sequence
  Kappa fragment
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 578 aa, Artificial Sequence
  VL1-Linker-VH1-CH1-CH2-CH3(Knob)
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 573 aa, Artificial Sequence
  VL2-Linker-VH2-CH1-CH2-CH3(Hole)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 11 - protein, 106 aa, Artificial Sequence
  Kappa fragment
CVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 578 aa, Artificial Sequence
  VL1-Linker-VH1-CH1-CH2-CH3(Knob)
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRCGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 573 aa, Artificial Sequence
  VL2-Linker-VH2-CH1-CH2-CH3(Hole)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRCGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 14 - protein, 699 aa, Artificial Sequence
  VH1-Linker-VL1-CL-Linker-CH1-CH2-CH3(Knob)
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 694 aa, Artificial Sequence
  VH2-Linker-VL2-CL-Linker-CH1-CH2-CH3(Hole)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 946 aa, Artificial Sequence
  VH1-Linker-VL1-CL-Linker-CH1-Linker-VH2- Linker-VL2-CL-Linker-CH1
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 17 - protein, 705 aa, Artificial Sequence
  VL1-linkerMT(G1C)-VH1-CH1-linker-VL2-linkerMT (G1C)-VH2-CH1
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRCGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRCGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2019/0309092 A1
(43) Pub. Date: Oct. 10, 2019

(54) MODIFIED ANTIGEN-BINDING FAB FRAGMENTS AND ANTIGEN-BINDING MOLECULES COMPRISING THE SAME

(71) Applicants: DCB-USA LLC, Wilmington, DE (US); Development Center for Biotechnology, New Taipei City (TW)

(72) Inventors: Chih-Yung HU, New Taipei City (TW); Chao-Yang HUANG, New Taipei City (TW); Yu-Jung CHEN, New Taipei City (TW); Chia-Cheng WU, New Taipei City (TW); Chien-Tsun KUAN, New Taipei City (TW); Chia-Hsiang LO, New Taipei City (TW); Hsien-Yu TSAI, New Taipei City (TW)

(73) Assignee: Development Center for Biotechnology, New Taipei City (TW)

(21) Appl. No.: 16/318,615

(22) PCT Filed: Jul. 20, 2017
(86) PCT No.: PCT/US17/43126

§ 371 Date: Jan. 17, 2019

Related U.S. Application Data
(60) Provisional application No. 62/364,854, filed on Jul. 21, 2016.

(51) Int. Cl. C07K 16/46; C07K 16/18; C07K 16/30; C07K 16/22; C07K 16/28; C07K 16/24; C07K 16/32

(52) U.S. Cl. CPC C07K 16/468; C07K 16/18; C07K 16/3084; C07K 16/22; C07K 16/2839; C07K 16/241; C07K 2317/92; C07K 2317/55; C07K 2317/522; C07K 2317/31; C07K 2317/526; C07K 2317/524; C07K 16/32

(57) ABSTRACT

The present invention provides a modified antigen-binding Fab fragment. An antigen-binding molecule comprising the antigen-binding Fab fragment and a composition comprising the molecule are also provided.

### CROSS-REFERENCE TO RELATED APPLICATION

This application claims priority to U.S. Provisional Application No. 62/364,854 filed Jul. 21, 2016; the disclosure of which is incorporated herein by reference in its entirety.

### FIELD OF THE INVENTION

The present invention relates to modified antigen-binding Fab fragments, comprising a light chain variable domain (VL), a light chain constant domain (CL), a heavy chain variable domain (VH) and a heavy chain constant domain 1 (CH1), wherein the C-terminus of the VL domain is linked via a linker to the N-terminus of the VH domain, or the C-terminus of the VII domain is linked via a linker to the N-terminus of the VL domain.

### BACKGROUND OF THE INVENTION

A bispecific antibody (BsAb) is an artificial protein that can simultaneously bind to two different types of antigen. Therapeutic monoclonal antibodies (Mabs) are widely used to treat human diseases. However, targeting only one antigen is usually insufficient for indications like cancer and relapse often occurs. Due to this phenomenon, an increasing number of combination therapies targeting existing biomarkers are under investigation.

In 2014, the US Food and Drug Administration's accelerated approval of Blinatumomab (Amgen Inc.) marks the first US approval for the BsAb. The FDA's approval of BsAb inspires more and more researchers on the extensive investigations of BsAb for the treatment of cancers, infectious diseases, other diseases or disorders.

BsAb can be manufactured in many structural formats, such as “IgG-like BsAb” or “non-IgG-like BsAb.” “Non-IgG-like BsAb.” for example, can be chemically linked antigen-binding Fab fragment consisting of only the Fab regions, or various types of bivalent and trivalent single-chain variable fragments (scFvs). “IgG-like BsAb” comprises two Fab arms and one Fc region, except the two Fab sites bind different antigens. IgG-like BsAb can be symmetric or asymmetric, depending on whether two heavy chains are identical.

BsAbs can be designed to simultaneously bind a cytotoxic cell (using a receptor like CD3) and a target such as a cancer cell (e.g., an antigen on the cancer cell). Such BsAbs can engage cytotoxic T cells for T cell-mediated cytotoxicity against defined target cells (e.g., cancer cells). One approach to BsAb (or a bispecific binding molecule) design is to have the two binding domains attach to the two ends (the N-terminal and the C-terminal ends) of a bridging domain (such as a constant region of an antibody). An example of such an approach is disclosed in the U.S. Patent Application Publication No 2013/0165638A1. An alternative approach to bispecific antibody construction (or a bispecific binding molecule) is to have different binding domains occupying the two antigen-binding sites on a natural IgG antibody.

The generation of asymmetric BsAb can be achieved by adopting the knobs-into holes (KiH) strategy (U.S. Pat. No. 7,695,936 B2). Specifically, the concept of KiH strategy relies on modifications of the interface between the two CH3 domains. A bulky residue is introduced into the CH3 domain of one antibody heavy chain and acts similarly to a key. In the other heavy chain, a “hole” is formed that is able to accommodate the bulky residue, mimicking a lock. The resulting heterodimeric Fc-part can be further stabilized by artificial disulfide bridges.

One drawback of KiH strategy is that there is still a random association with the light chains (i.e. light chain mispairing, see FIG. 1). The issue of light chain mispairing can be addressed by generating bispecific molecules with common light chains (Merchant A M et al. An efficient route to human bispecific IgG; Nat Biotechnol 1998; 16:677-81) or by domain swapping between one heavy and light chain resulting in CrossMabs (Schaefer W et al. Immunoglobulin domain crossover as a generic approach for the production of bispecific IgG antibodies: Proc Natl Acad Sci USA 2011; 108:11187-92).

U.S. Pat. No. 9,382,323 B2 relates to a bispecific antibody comprising “a full length antibody” and “one or two single chain Fab fragments” fused to the full-length antibody via a peptide connector at the C- or N-terminus of the heavy chain of the full-length antibody. However, the full length antibody of U.S. Pat. No. 9,382,323 B2 is a symmetric antibody comprising two identical heavy and light chains and is monovalent against one antigen. U.S. Pat. No. 9,382,323 B2 neither mentions the issue of light chain mispairing in the preparation of a bispecific antibody, nor the technical means for addressing light chain mispairing.

There is still a need to develop a novel strategy to overcome light chain mispairing issue in the preparation of asymmetric BsAb while retaining strong affinty to the antigen(s) specifically.

### SUMMARY OF THE INVENTION

The present invention provides a mean to modify the structure of a Fab region to reduce the mispairing rate during the formation of an antigen-binding molecule and improve the production of the molecule.

Therefore, one aspect of the invention is to provide an antigen-binding Fab fragment, comprising a light chain variable domain (VL), a light chain constant domain (CL), a heavy chain variable domain (VH) and a heavy chain constant domain 1 (CH1), wherein the C-terminus of the VL domain is linked via a linker to the N-terminus of the VH domain; or the C-terminus of the VH domain is linked via a linker to the N-terminus of the VL domain.

In a preferred embodiment of the antigen-binding Fab fragment of the invention, the C-terminus of the VL domain is linked via a linker to the N-terminus of the VH domain, and wherein (1) the C-terminus of the VH domain is linked to the N-terminus of the CH1 domain through a peptide bond: (2) the C-terminus of the VH domain is linked to the N-terminus of the CL domain through a peptide bond; (3) the C-terminus of the VII domain is linked to the N-terminus of the CL domain through a peptide bond, and the C-terminus of the CL domain is linked via a linker to the N-terminus of the CH1 domain; or (4) the C-terminus of the VH domain is linked to the N-terminus of the CH1 domain through a peptide bond, and the C-terminus of the CH1 domain is linked via a linker to the N-terminus of the CL domain.

In another preferred embodiment of the antigen-binding Fab fragment of the invention, the C-terminus of the VH domain is linked via a linker to the N-terminus of the VL domain, and wherein (1) the C-terminus of the VL domain is linked to the N-terminus of the CH1 domain through a peptide bond; (2) the C-terminus of the VL domain is linked to the N-terminus of the CL domain through a peptide bond; (3) the C-terminus of the VL domain is linked to the N-terminus of the CL domain through a peptide bond, and the C-terminus of the CL domain is linked via a linker to the N-terminus of the CH1 domain; or (4) the N-terminus of the VL domain is linked to the C-terminus of the CH1 domain through a peptide bond, and the N-terminus of the CH1 domain is linked via a linker to the C-terminus of the CL domain.

In a further preferred embodiment of the antigen-binding Fab fragment of the invention, (1) the VL domain is linked to the CL domain through a disulfide bond; (2) the VH domain is linked to the CL domain through a disulfide bond; (3) the VL domain is linked to the CH1 domain through a disulfide bond; or (4) the VH domain is linked to the CH1 domain through a disulfide bond.

In a further embodiment of the invention, the antigen-binding Fab fragment optionally comprises an Fc region.

Another aspect of the invention is to provide an antigen-binding molecule comprising two or more of the antigen-binding Fab fragments of the present invention, wherein the two or more fragments are specific to identical or different antigens.

Another aspect of the invention is to provide a polynucleotide encoding the antigen-binding Fab fragment of the present invention.

Another aspect of the invention is to provide vectors and host cells for expressing the antigen-binding Fab fragments or antigen-binding molecules of the present invention.

Another aspect of the invention is to provide a method for preparing the antigen-binding molecule of the present invention.

Another further aspect of the invention is to provide a pharmaceutical composition comprising the antigen-binding molecule of the present invention.

The present invention is described in detail in the following sections. Other characterizations, purposes and advantages of the present invention can be easily found in the detailed descriptions and claims of the invention.

### BRIEF DESCRIPTION OF THE DRAWINGS

FIG. 1 refers to schematic diagram of the possible products of IgG BsAb assembly.

FIG. 2A refers to conformation of native Fab. FIGS. 2B to 2I refer to the possible conformations of the Fab fragment of the invention.

FIGS. 3A to 3Q refer to the possible conformations of the antigen-bind molecules of the invention.

FIGS. 4A to 4E refer to the nucleic acid sequences for encoding the antigen-binding molecules of the invention. “*” denotes the introduction of knobs-into-holes into the CH3 domains. “#” denotes the introduction of an engineered-cysteine into the VL1 domain, VL2 domain and CL domain. “P,” “P1” and “P2” denote promoter.

FIGS. 5A to 5H refer to the vectors for expression of the antigen-bind molecules of the invention.

FIGS. 6A to 6N refer to SDS-PAGE results of the BsAb clones obtained from Example 2.

### DETAILED DESCRIPTION OF THE INVENTION

### Definitions

Unless otherwise defined herein, scientific and technical terms used in connection with the present invention shall have the meaning commonly understood by those of ordinary skill in the art. The meaning and scope of the terms should be clear; however, in the event of any latent ambiguity, definitions provided herein take precedence over any dictionary or extrinsic definition.

Unless otherwise required by context, singular terms shall include the plural and plural terms shall include the singular. For example, the term “a” or “an,” as used herein, is defined as one or more than one.

As used herein, the term “or” in the claims is used to mean “and/or” unless explicitly indicated to refer to alternatives only or the alternatives are mutually exclusive.

As used herein, the terms “first,” “second,” etc. refer to different units (for example, a first nucleic acid, a second nucleic acid). The use of these terms herein does not necessarily connote an ordering such as one unit or event occurring or coming before another, but rather provides a mechanism to distinguish between particular units.

As used herein, the term “antigen-binding Fab fragment” refers to a fragment comprising a heavy chain variable domain (VH), a light chain variable domain (VL), a heavy chain constant domain (CH) and a light chain constant domain (CL). Each Fab fragment is monovalent with respect to antigen binding. The Fab fragment may further comprise a Fab region (containing CH2 and CH3 domains) of an IgG antibody.

As used herein, the term “antigen-binding molecule” refers to a molecule that specifically binds one or more antigens. Examples of antigen-binding molecules are IgG-like molecules or non-IgG-like molecules.

As used herein, the term “single-chain variable fragment (scFv)” refers to a fusion protein of the VH and VL of immunoglobulins connected with a linker, which can connect the N-terminus of the VH with the C-terminus of the VL, or connect the N-terminus of the VL with the C-terminus of the V H.

As used herein, the term “cysteine-engineered antibody” refers to an antibody comprising one or more cysteine residues that are not normally present at a native antibody light chain or heavy chain. Such cysteine residue is thus referred to as “engineered cysteine.” The engineered cysteine can be introduced by using conventional technologies such as those in Molecular Immunology, Vol. 32, NO. 4, pp. 249-258, 1995.

As used herein, the term “polynucleotide” or “nucleic acid” refers to polymers of nucleotides, and may be in the form of DNA or RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction.

As used herein, the term “vector” refers to a nucleic acid molecule capable of transporting another nucleic acid sequence to which it has been linked. The vector may be a “plasmid.” which refers to a circular double-stranded DNA loop into which additional DNA segments may be introduced.

As used herein, the term “transfection” refers to the process of introducing a polynucleotide into eukaryotic cells.

As used herein, the term “promoter” refers to a region of DNA that initiates transcription of a particular gene. Promoters are located near the transcription start sites of genes, on the same strand and upstream on the DNA (towards the 5′ region of the sense strand).

As used herein, the term “modification” refers to a change of an amino acid sequence as compared to an original amino acid sequence. The modifications include, for example, substitution of an amino acid residue with another amino acid, insertion of one or more amino acids, and deletion of amino acid residue(s).

As used herein, the term “pharmaceutical composition” refers to a formulation or preparation comprising an active ingredient having biological or pharmacological activity and a pharmaceutically acceptable carrier. The pharmaceutical composition may be in the form of tablets, powder, pellets, beads, granules, microspheres, capsule, pills and so forth.

As used herein, the term “pharmaceutically acceptable carrier” refers to solvents, diluents, binders, adhesives, adjuvants, excipients, acceptors, stabilizers, analogues, flavoring agents, sweetening agents, emulsifying agents and/or preservative agents, which are well known to in the art, for manufacturing pharmaceutical compositions. Examples of the pharmaceutically acceptable carrier include, but are not limited to water, saline, buffers, inert, and nontoxic solids.

General Techniques

The present invention can use conventional techniques of molecular biology, chemistry, biochemistry, cell biology, microbiology and immunology. The prior art literature/references that describe the conventional techniques, include Molecular Cloning: A Laboratory Manual (Fourth Edition). Monoclonal Antibodies: A Practical Approach (First Edition) and Current Protocols in Molecular Biology.

Cell Culture Conditions

Host Cells

The Fab fragments or antigen-binding molecules of the invention can be produced in host cells, including prokaryotic, eukaryotic and plant host cells. The prokaryotic host cell, for example, may be Escherichia coli (E. coli) The eukaryotic host cells that are suitable for producing the Fab fragments or antigen-binding molecules of the invention include, but are not limited to, African green monkey kidney (COS) cells. Chinese hamster ovary (CHO) cells, myeloma cells (such as SP 2/0, YB 2/0. NS0 and P3X63. Ag8.653), baby hamster kidney (BHK) cells, human embryonic kidney (HEK-293) cells, Freestyle 293 cells and human retina-derived PER-C6 cells (PER.C6® human cells). In a preferred embodiment of the invention, the cells are CHO cells or Freestyle 293 cells.

Promoters

To achieve high levels of expression of antigen-binding molecules of the invention, strong promoters are used to drive antibody heavy chain and light chain expression. The promoters can be eukaryotic promoters or prokaryotic promoters. The prokaryotic promoters suitable for production of the Fab fragments or antigen-binding molecules of the invention include, but are not limited to T7, T7lac, Sp6, araBAD, trp, lac, Ptac and pL. The eukaryotic promoters suitable for production of the Fab fragments or antigen-binding molecules of the invention include, but are not limited to cytomegalovirus (CMV), elongation factor alpha (EF1α). SV40, PGK1, Ubc, human beta actin, CAG, TRE, UAS, Ac5, Polyhedrin, CaMKIIa, GAL1, GAL10, TEF1, GDS, ADH1, CaMV35S, Ubi, H1 and U6. In a preferred embodiment of the invention, the promoter used is CMV promoter.

Vector Design

In one embodiment of the invention, the vector contains a nucleic acid sequence encoding a VL-linker-VH-CH1 or VH-linker-VL-CH1 segment and a nucleic acid sequence encoding the CL domain, wherein the expression of the two nucleic acid sequences is driven by two promoters, respectively. In another embodiment of the invention, the nucleic acid sequence encoding the VL-linker-VH-CH1 or VH-linker-VL-CH1 segment further includes a nucleic acid sequence encoding an Fc region (including CH2 domain and CH3 domain) at 3′-end.

In one embodiment of the invention, the vector contains a nucleic acid sequence encoding a VL-linker-VH-CL or VH-linker-VL-CL segment and a nucleic acid sequence encoding the CH1 domain, wherein expression of the two nucleic acid sequences is driven by two promoters, respectively. In another embodiment of the invention, the nucleic acid sequence encoding the VL-linker-VH-CL and VH-linker-VL-CL segments further include an nucleic acid sequence encoding an Fc region (including CH2 domain and CH3 domain) at 3′-end.

In one embodiment of the invention, the vector contains a nucleic acid sequence encoding a VH-linker-VL-CL-linker-CH1, VL-linker-VH-CL-linker-CH1, VH-linker-VL-CH1-linker-CL or VL-linker-VH-CH1-linker-CL segment. In another embodiment of the invention, the nucleic acid sequences encoding the VH-linker-VL-CL-linker-CH1. VL-linker-VH-CL-linker-CH1. VH-linker-VL-CH1-linker-CL and VL-linker-VH-CH1-linker-CL segments further include an nucleic acid sequence encoding an Fc region (including CH2 domain and CH3 domain) at 3′-end.

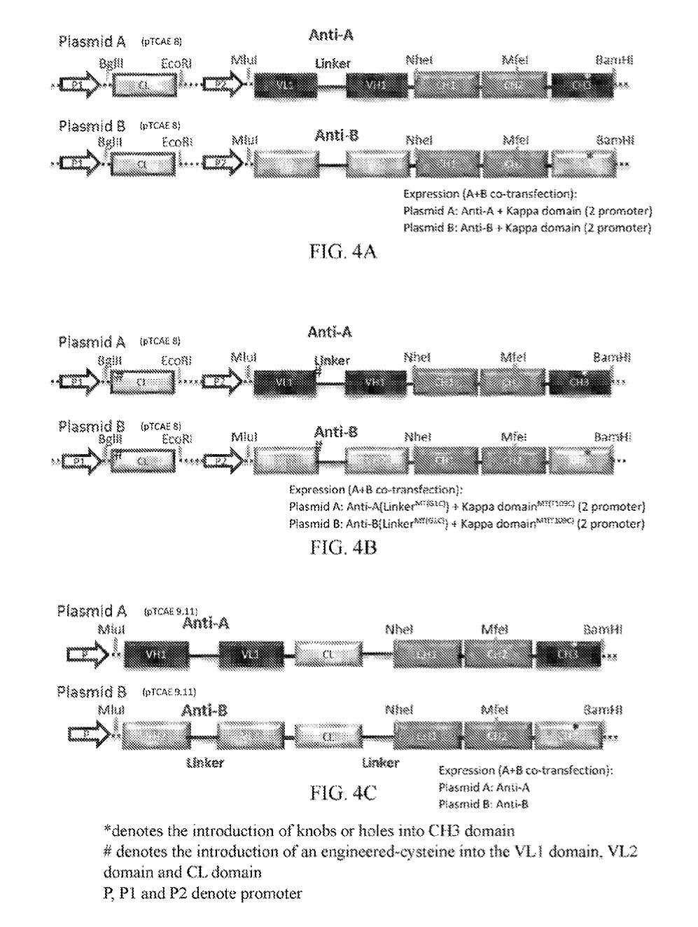

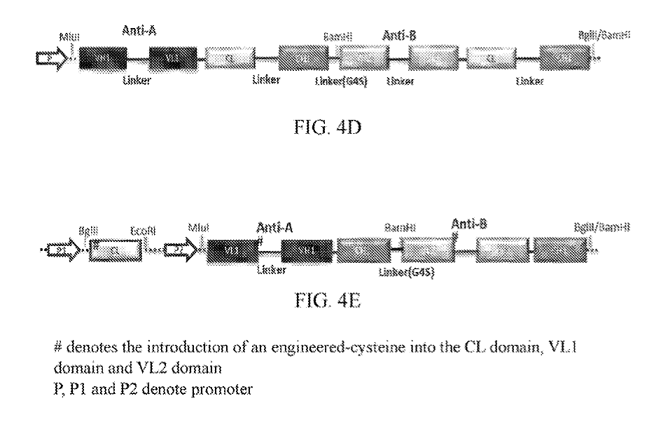

In one embodiment of the invention, two or more vectors are transfected to a host cell to produce the antigen-binding molecule of the present invention (e.g., FIGS. 4A to C). The vectors respectively express antigen-binding fragments specific to different antigens.

The Sequence and Length of Linker Used in the Invention

Any linkers known in the art can be used in the invention. The linker can be a peptide of 3 to 50 amino acids, preferably 5 to 40 amino acids, more preferably 5 to 30 amino acids, even more preferably 10 to 25 amino acids, and most preferably 15 amino acids. The linker is usually rich in glycine for flexibility, as well as serine or threonine for solubility. Table 1 shows the sequences and lengths of the example linkers that can be used in the invention (Biotechnology and Genetic Engineering Reviews, 2013, Vol. 29, No. 2, 175-186). In an embodiment of the invention, the linker has the sequence of GGGGSGGGGSGGGGS (SEQ ID NO: 1).

TABLE 1

| Length | Amino Acid Sequence | SEQ ID NO: |
|---|---|---|
| 15 | GGGGSGGGGSGGGGS | 1 |
| 15 | SGGGSGGGGSGCGCS | 2 |
| 18 | GSTSGSGKPGSGEGSTKG | 3 |
| 26 | SSADDAKKDAAKKDDAKKDDAKKDAS | 4 |
| 16 | GGGGSGGGGSCGGGGS | 5 |
| 14 | EGKSSGSGSESKVD | 6 |
| 16 | DGGGSGGGGSGGGGSS | 7 |

Conformations of the Fab Fragment and Antigen-Binding Molecule of the Invention

The present invention provides a series of Fab fragments that are different from the known Fab in conformation.

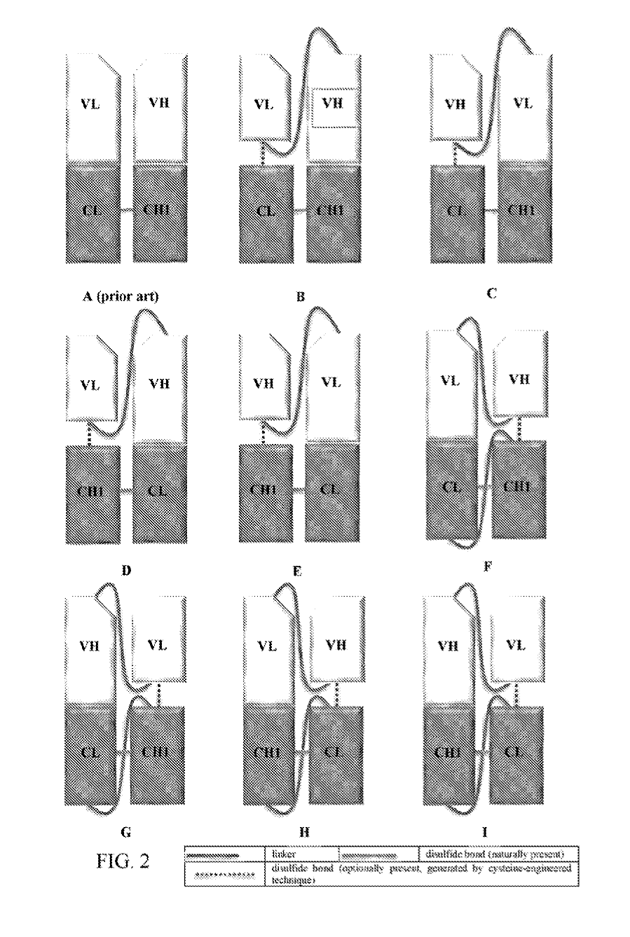

As shown in FIG. 2A, n a native Fab, the C-terminus of VL is linked to the N-terminus of CL through a peptide bond, the C-terminus of VH is linked to the N-terminus of CH1 through a peptide bond, and CL is linked to CH1 via a disulfide bond.

One aspect of the invention is to provide a modified Fob fragment comprising:
(a) a VL domain, a CL domain, and a VH-CH1 region, wherein the C-terminus of the VL domain is linked via a linker to the N-terminus of the VH domain, and wherein the VL domain is optionally linked to the CL domain via a disulfide bond (FIG. 2B);
(b) a VH domain, a CL domain, and a VL-CH1 region, wherein the C-terminus of the VH domain is linked via a linker to the N-terminus of the VL domain, and wherein the VH domain is optionally linked to the CL domain via a disulfide bond (FIG. 2C);
(c) a VL domain, a CH1 domain, and a VH-CL region, wherein the C-terminus of the VL domain is linked via a linker to the N-terminus of the VH domain, and wherein the VL domain is optionally linked to the CH1 domain via a disulfide bond (FIG. 2D);
(d) a VH domain, a CH1 domain, and a VL-CL region, wherein the C-terminus of the VH domain is linked via a linker to the N-terminus of the VL domain, and wherein the VH domain is optionally linked to the CH1 domain via a disulfide bond (FIG. 2E).
(e) a VH, a CH1, and a VL-CL region, wherein the C-terminus of the VH domain is linked via a first linker to the N-terminus of the VL domain; and the N-terminus of the CH1 domain is linked via a second linker to the C-terminus of the CL domain, wherein the first linker and the second linker are the same or different, and wherein the VH domain is optionally linked to the CH1 domain via a disulfide bond (FIG. 2F);
(f) a VL, a CH1, and a VH-CL region, wherein the C-terminus of the VL domain is linked via a first linker to the N-terminus of the VH domain; and the N-terminus of the CH1 domain is linked via a second linker to the C-terminus of the CL domain, wherein the first linker and the second linker are the same or different, and wherein the VL domain is optionally linked to the CH1 domain via a disulfide bond (FIG. 2G);
(g) a VH, a CL, and a VL-CH1 region, wherein the C-terminus of the VH domain is linked via a first linker to the N-terminus of the VL domain; and the N-terminus of the CL domain is linked via a second linker to the C-terminus of the CH1 domain, wherein the first linker and the second linker are the same or different, and wherein the VH domain is optionally linked to the CL domain via a disulfide bond (FIG. 2H); or
(h) a VL, a CL, and a VH-CH1 region, wherein the C-terminus of the VL domain is linked via a first linker to the N-terminus of the VH domain; and the N-terminus of the CL domain is linked via a second linker to the C-terminus of the CH1 domain, wherein the first linker and the second linker are the same or different, and wherein the VL domain is optionally linked to the CL domain via a disulfide bond (FIG. 2I).

The disulfide bond can be formed by employing the cysteine-engineered technique as described herein. The Fab fragment or antigen-binding molecule of the invention having such disulfide bond is referred to as “disulfide-bond stabilized” format.

In another embodiment of the invention, each of the VH-CH1, VL-CH1, VH-CL, and VL-CL regions may further include an Fc region.

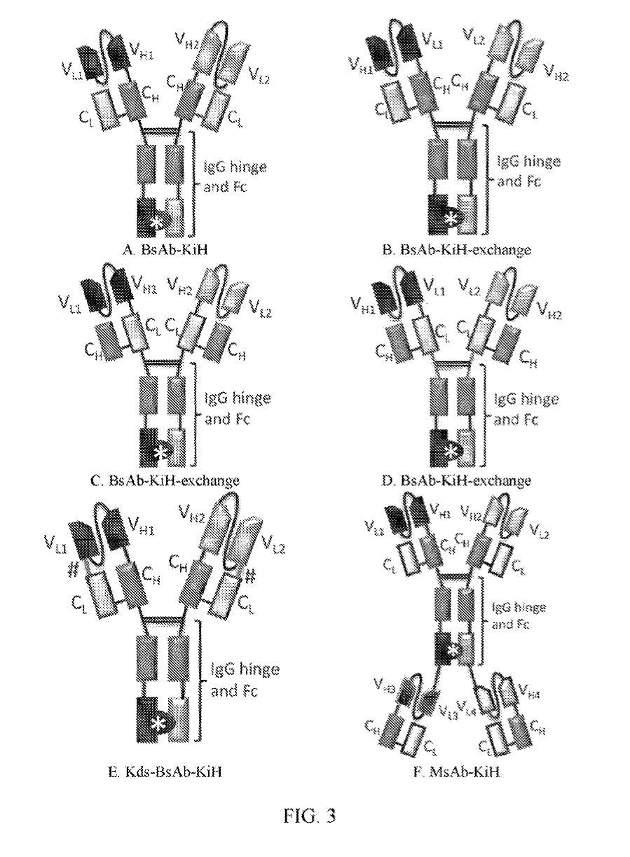

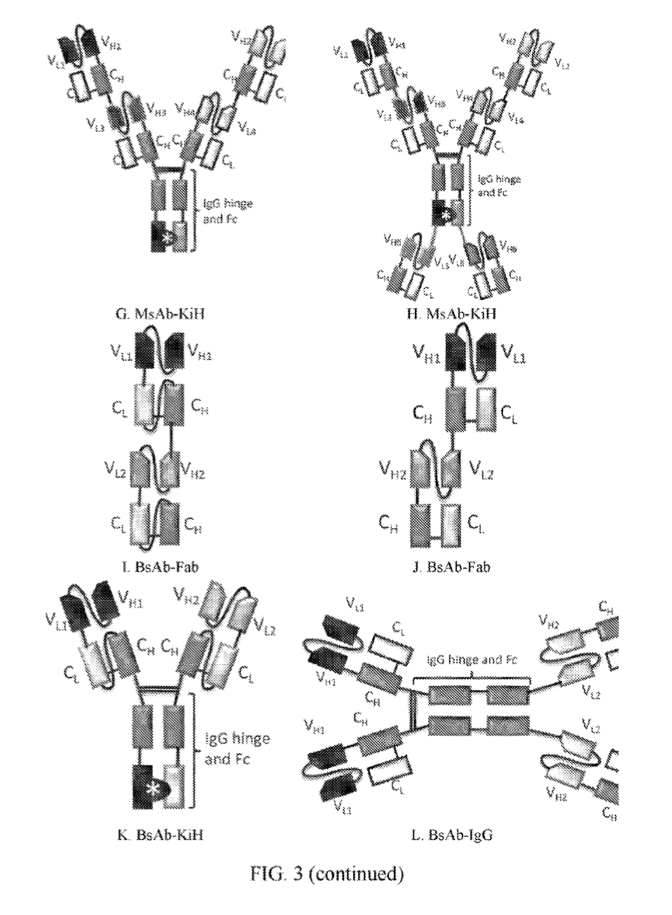

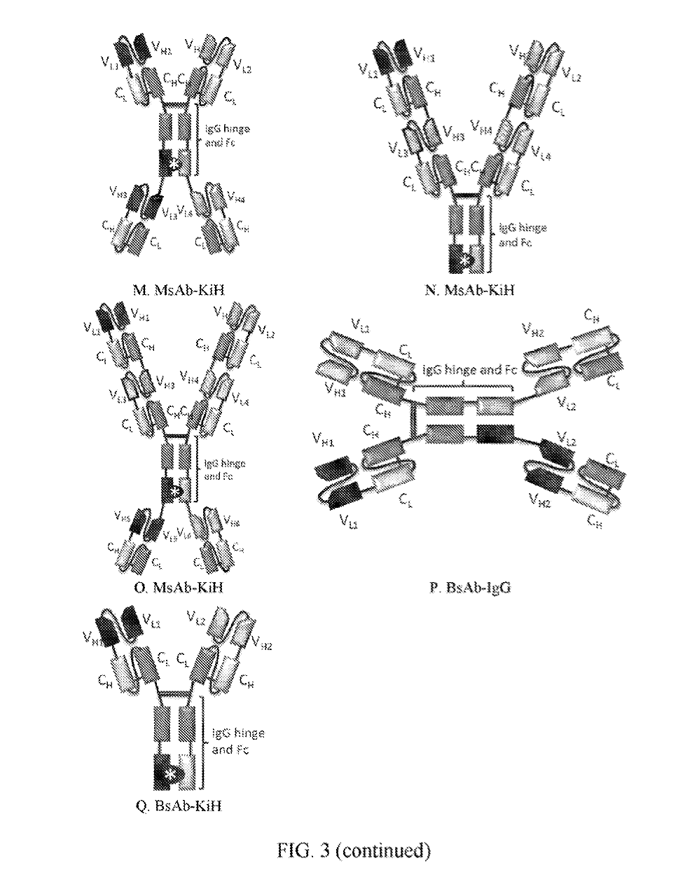

In view of the modified Fab fragments of the invention, examples of the conformations of the antigen-binding molecule may include those shown in FIGS. 3A-K, and the antigen-binding molecules of the present invention may be mono-specific or bispecific (Roland Kontenman (2012) Dual targeting strategies with bispecific antibodies, mAbs, 4:2, 182-197).

In an embodiment of the invention, any strategies known in the art, such as KiH strategy and those disclosed in Christian Klein et al. (“Progress in overcoming the chain association issue in bispecific heterodimeric IgG antibodies,” MAbs. 2012 Nov. 1; 4(6): 653-663), can be used in the formation of the antigen-binding molecules having a Fc region. For example, in the KiH strategy, the locations of the nucleic acid sequences of the two CH3 domains in the two Fc regions can be modified as shown in Table 2.

TABLE 2

| Modification location(s)* in CH3 | Modification location(s)* in |
|---|---|
| of HC1 to create a protuberance | other CH3 of HC 2 to create a |
| (knob) | cavity (hole) |
| T366Y | Y407A |
| T366W | Y407A |
| T366Y | Y407T |
| T394W | F405A |
| T366Y/F405A | T394W/Y407T |
| T366W/F405W | T394S/Y407A |
| F405W | T394S |
| D399C | K392C |
| T366W | T366S/L368A/Y407V |
| T396W/D399C | T366S/L368A/K392C/Y407V |
| T366W/K392C | T366S/D399C/L368A/Y407V |
| S354C/T366W | Y349C/T366S/L368A/Y407V |
| Y349C/T366W | S354C/T366S/L368A/Y407V |
| E356C/T366W | Y349C/T366S/L368A/Y407V |
| Y349C/T366W | E356C/T366S/L3668A/Y407V |
| E357C/T366W | Y349C/T366S/L368A/Y407V |
| Y349C/T366W | E357C/T366S/L368A/Y407V |
| K370E/D399K/K439D | D356K/E357K/K490D |
| K409D | D399K |
| K409E | D399K |
| K409E | D399R |
| K409D | D399R |
| D339K | E356K |
| D399K/E356K | K409D/K392D |
| D399K/E356K | K409D/K469D |
| D399K/E357K | K409D/K370D |
| D399K/E356K/E357K | K409D/K392D/K370D |
| D399K/E357K | K409D/K392D |
| K392D/K409D | D399K |
| K409D/K360D | D399K |

*The numbering is according to the EU index.

Method for Preparing the Modified Fab Fragments or Antigen-Binding Molecules of the Invention

One aspect of the invention is to provide methods for preparing the Fab fragments and antigen-binding molecules of the subject application. The method may comprise the step of incubating a host cell as described above at a condition suitable for expression of the Fab fragment or antigen-binding molecule of the present invention. According to the invention, the host cell may comprise one or more vectors as described above. The condition suitable for expression of the Fab fragment and antigen-binding molecule may vary depending from the species of the promoter, vector and host cell used, and can be determined on the basis of prior art.

The following examples are provided to aid those skilled in the art in practicing the present invention. Even so, the examples should not be construed to unduly limit the present invention as modifications thereto, and variations on the embodiments discussed herein may be made by those having ordinary skill in the art without departing from the spirit or scope of the present inventive discovery.

### Example 1: Preparation of Vectors for Expression of the Antigen-Binding Molecule of the Invention

1. Method of Generating the Antigen-Binding Molecule of FIG. 3A

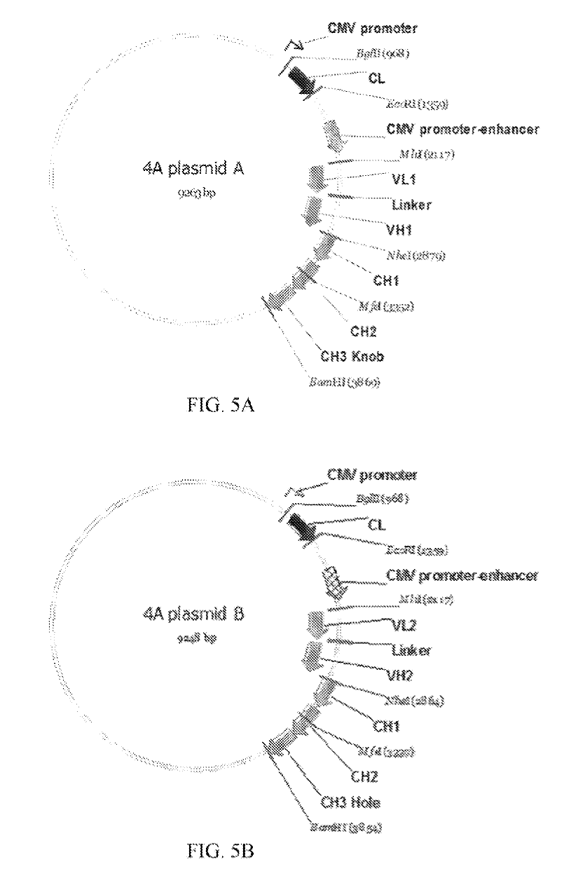

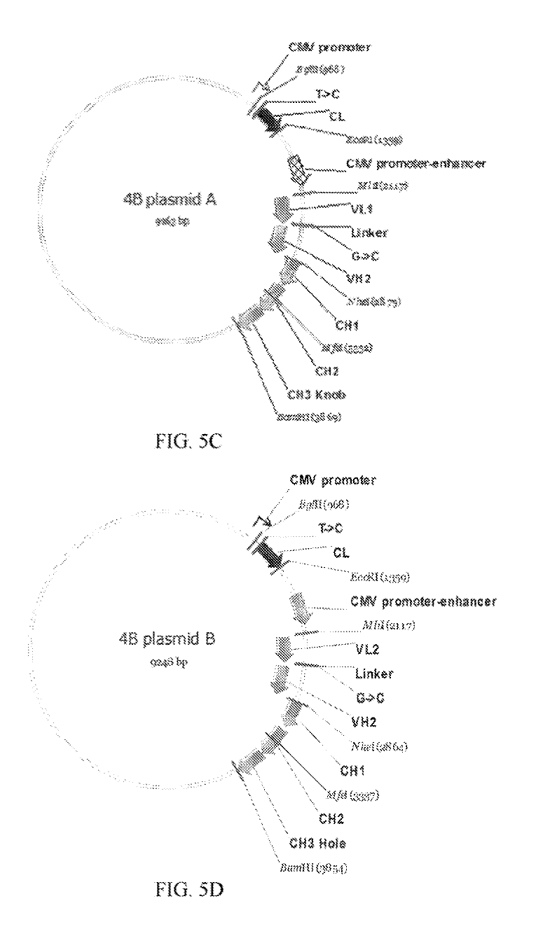

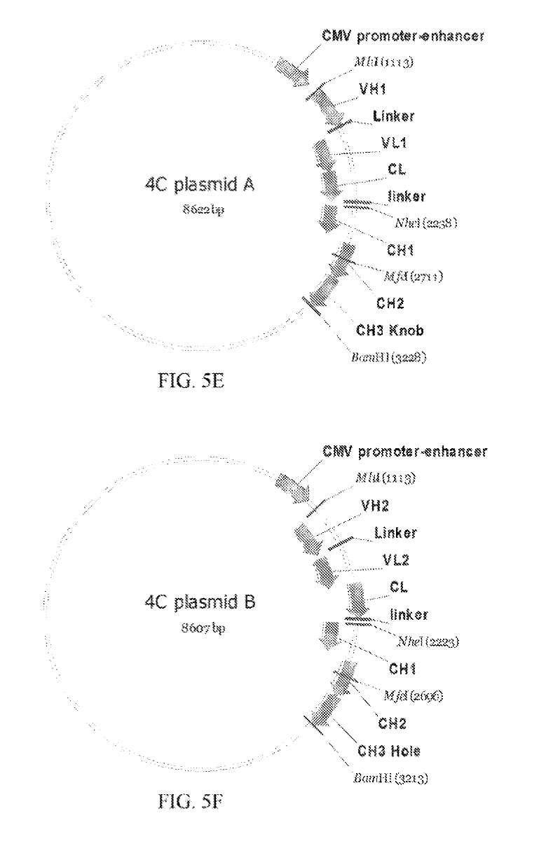

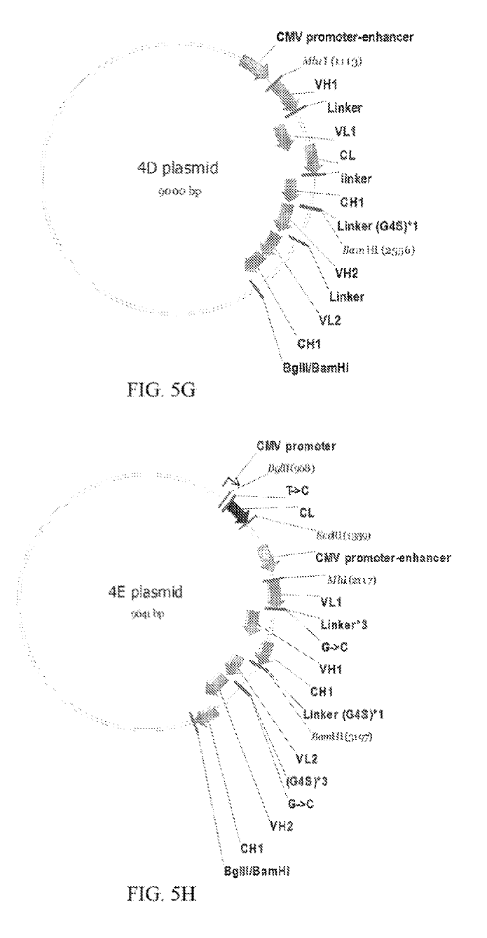

Conformations of the polynucleotide sequences encoding the antigen-binding molecule of FIG. 3A are shown in FIG. 4A, and the expression vectors are shown in FIGS. 5A and 5B.

Two polynucleotide sequences encoding two VL-linker-VH-CH1-CH2 fragments (“VL1-Linker-VH1-CH1-CH2-CH3 (Knob)” and “VL2-Linker-VH2-CH1-CH2-CH3 (Hole)”) specific to two different antigens were generated by gene synthesis. The two polynucleotide sequences were subcloned into an antibody expressing plasmid pTACE8 after MluI and MfeI restriction enzyme digestion. The linker has the amino acid sequence of GGGGSGGGGSGGGGS (SEQ ID NO: 1).

Two polynucleotide sequences encoding two CH3 domains were modified by PCR amplification to include the knob arm genes S354C and T366W in the sequence of one CH3 domain; and include the hole arm genes Y349C, T366S, L368A, and Y407V in the sequence of the other CH3 domain. The two modified polynucleotide sequences were subcloned into an antibody expressing plasmid pTACE8 after MfeI and BamHI restriction enzyme digestion to form the VL-linker-heavy chain Knob or Hole.

A polynucleotide sequence encoding the Kappa fragment was generated by gene synthesis. The polynucleotide sequence was subcloned into an antibody expressing plasmid pTACE8 that include the VL-linker-heavy chain Knob or Hole arms after Bgl II and EcoRI restriction enzyme digestion.

The amino acid sequences are shown in Table 3

TABLE 3

| | Amino Acid Sequence | SEQ ID NO: |
|---|---|---|
| Kappa fragment | TVAAPSVFIFPPSDEQLKSGTASVVCLLNNFYPREAKV | 8 |
| | QWKVDNALQSGNSQESVTEODSKDSTYSLSSTLTLSK | |
| | ADYEKHKVYACEVTHQGLSSPVTKSFNRGEC | |
| 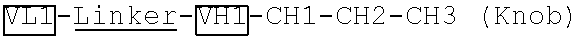 | 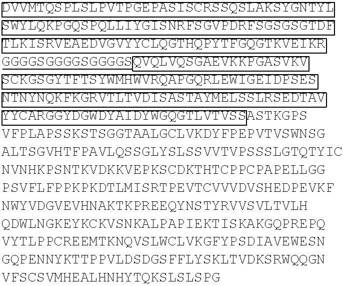 | 9 |
| 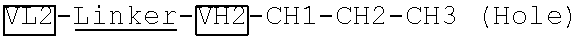 | 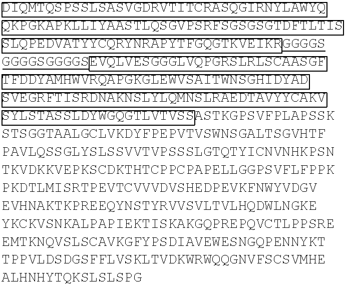 | 10 |

II. Method of Generating the Antigen-Binding Molecule of FIG. 3E

Conformations of the polynucleotide sequences encoding the antigen-binding molecule of FIG. 3E are shown in FIG. 4B, and the expression vectors are shown in FIGS. 5C and 5D.

Two plasmids from 4A encoding two VL-linker-VH-CH1-CH2-CH3 fragments (“VL1-Linker-VH1-CH1-CH2-CH3 (Knob)” and “VL2-Linker-VH2-CH1-CH2-CH3 (Hole)”), which are specific to two different antigens and have Knob-and-Hole modifications, were generated by the method described above. The linker has the amino acid sequence of GGGGSGGGGSGGGGS (SEQ ID NO: 1).

Two polynucleotide sequences encoding two VL-cysteine mutant linker-VH fragments specific to two different antigens were generated by gene synthesis. The two polynucleotide sequences containing engineered cysteine were subcloned into an antibody expressing vector pTACE8 from 4A after MluI and NheI restriction enzyme digestion.

A polynucleotide encoding a Kappa fragment with an engineered cysteine was generated by gene synthesis. Said polynucleotide was subcloned into an antibody expressing vector after BglII and EcoRI restriction enzyme digestion.

The amino acid sequences are shown in Table 4

TABLE 4

| | Amino Acid Sequence | SEQ ID NO: |
|---|---|---|
| Kappa fragment | CVAAPSVFIFPPSDEQLKSGTASVVCLLNNFYPREAKV | 11 |
| | QWKVDNALQSGNSQESVTEQDSKDSTYSLSSTLTLSK | |
| | ADYEKHKVYACEVTHQGLSSPVTKSFNRGEC | |
| 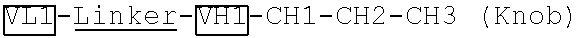 | 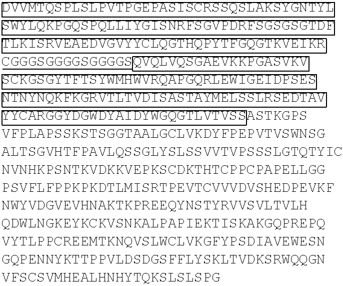 | 12 |
| 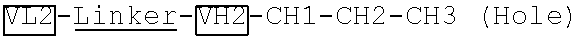 | 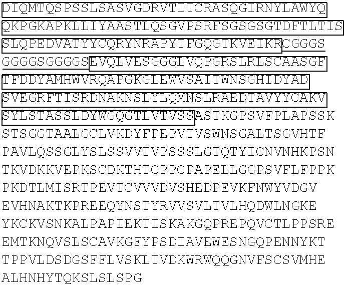 | 13 |

III. Method of Generating the Antigen-Binding Molecule of FIG. 3K

Conformations of the polynucleotide sequences encoding the antigen-binding molecule of FIG. 3K are shown in FIG. 4C, and the expression vectors are shown in FIGS. 5E and 5F.

Two polynucleotide sequences encoding two CH3 domains were modified by PCR amplification to include the knob arm genes S354C and T366W in the sequence of one CH3 domain; and include the hole arm genes Y349C, T366S, L368A, and Y407V in the sequence of the other CH3 domain. The two modified polynucleotide sequences s ere subcloned into an antibody expressing vector pTCAE9.11 after MfeI and BamHI restriction enzyme digestion.

Two polynucleotide sequences encoding two VH-linker 1-VL-CL-linker 2-CH1-CH2 fragments (“VH1-Linker-VL1-CL-Linker-CH1-CH2-CH3 (Knob)” and “VH2-Linker-VL2-CL-Linker-CH1-CH2-CH3 (Hole)”), which are specific to two different antigens, were generated by the synthesis method. The linkers 1 and 2 have the same amino acid sequence of GGGGSGGGGGSGGGGS (SEQ ID NO: 1). The two polynucleotide sequences were subcloned into an antibody expressing vector pTCAE9.11 after MluI and MfeI restriction enzyme digestion.

The amino acid sequences are shown in Table 5.

TABLE 5

| | Amino Acid Sequence | SEQ ID NO: |
|---|---|---|
| 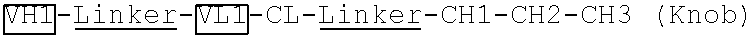 | 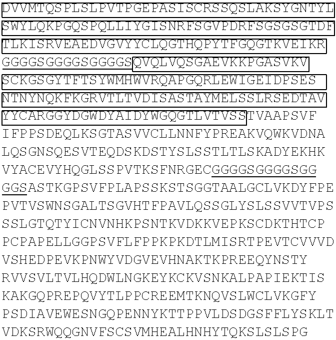 | 14 |
| 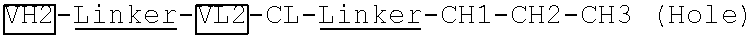 | 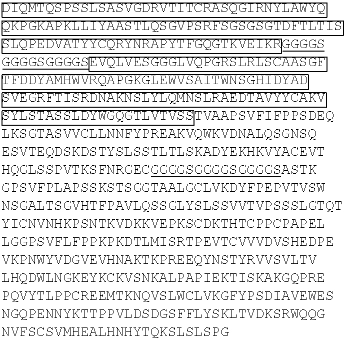 | 15 |

IV. Method of Generating the Antigen-Binding Molecule of FIG. 3I

Conformations of the polynucleotide sequences encoding the antigen-binding molecule of FIG. 3I are shown in FIG. 4D, and the expression vectors are shown in FIG. 5G.

VH1-Linker-VL1-CL-Linker-CH1-Linker-VH2-Linker-VL2-CL-Linker-CH1 fragment was generated by gene synthesis and was subcloned into an antibody expressing vector pTACE9.11 after MluI and BamHI restriction enzyme digestion. The linker has the amino acid sequence of GGGGSGGGGSGGGGS (SEQ ID NO. 1).

The amino acid sequences are shown in Table 6.

TABLE 6

| | | SEQ |
|---|---|---|
| | | ID |
| | Amino Acid Sequence | NO: |
| 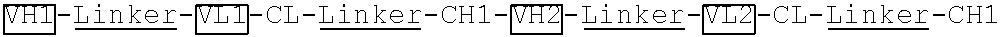 | 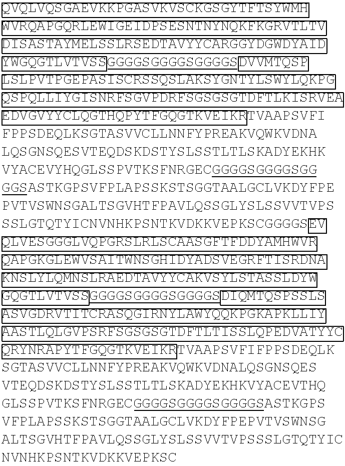 | 16 |

V. Method of Generating the Antigen-Binding Molecule of FIG. 3J

Conformations of the polynucleotide sequences encoding the antigen-binding molecule of FIG. 3J are shown in FIG. 4E, and the expression vectors are shown in FIG. 5H.

Kappa domain(CL)^{MT(T109C)} and VL1linker^{MT(G1C)}-VH1-CH1-linker-VL2-linker^{MT(G1C)-VH}2-CH1 were generated by gene synthesis. Kappa domain^{MT(T109C)} was subcloned into an antibody expressing vector pTACE9.11 after Bgl II and EcoRI restriction enzyme digestion. VL1-linker^{MT(G1C)}-CH1-linker-VL2-linker^{MT(G1C)}-VH2-CH1 was subcloned into the antibody expressing vector described above after MluI and Bgl II restriction enzyme digestion (vector was treated by MluI and BamHI restriction enzymes). The linker has the amino acid sequence of GGGGSGGGGSGGGGS (SEQ ID NO: 1).

The amino acid sequences are shown in Table 7.

TABLE 7

| | | SEQ |
|---|---|---|
| | | ID |
| | Amino Acid Sequence | NO: |
| Kappa domain (CL)^{MT(T109C)} | CVAAPSVFIFPPSDEQLKSGTASVVCLLNNFYPREAKV | 11 |
| | QWKVDNALQSGNSQESVTEQDSKDSTYSLSSTLTLSK | |
| | ADYEKHKVYACEVTHQGLSPVTKSFNRGEC | |
| 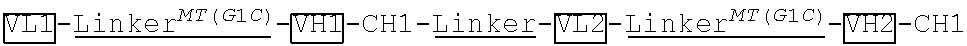 | 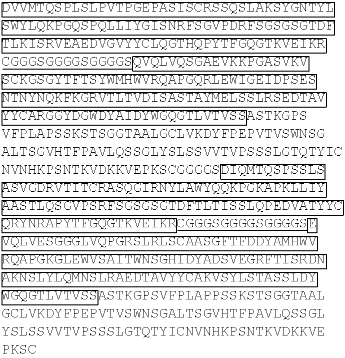 | 17 |

### Example 2: Expression and Purification of the Antigen-Binding Molecules of the Invention

Freestyle 293 cells were incubated in 15 mL Freestyle293 Expression medium at 37° C. and 8% CO₂ till a cell density of 2×10⁶ cell/mL. 37.5 μg of each of the antigen binding molecules expression vectors from Example 1 was incubated in 1.5 mL 150 mM NaCl as the vector solution, and 37.5 μl (2 mg/ml) PET (Polyethyleneimine) in 1.5 mL NaCl as the PEI/NaCl solution and sit at room temperature (RT) for 5 min. PEI/NaCl solution was added to the vector solution and stood at RT for 10 minutes as the vector/PEI mixed solution. The obtained vector/PEI mixed solution was added to Freestyle 293 cell preparation, and incubated at 37° C. and 8% CO₂ with shaking at 135-150 rpm for 4 hours. Fresh cell culture medium was added to the cells. Supernatant was collected and filtrated through a sterile filter after growing for 5-7 days. The antibodies were purified according to the manufacturer's protocol (MontageA). Table 8 shows the features of the antibodies obtained.

TABLE 8

| Clone No. | Construct Name | Target 1 | Target 2 | Format |
|---|---|---|---|---|
| 001 | XA-14 IgG (Full) | HSA | — | IgG(Native) |
| 002 | XA-14 IgG (LFv) | HSA | — | IgG(Fab*) |
| 003 | XA-14 scFv9.3 | HSA | — | scFv |
| 004 | XA-14 scFv9.5 | HSA | — | scFv |
| 005 | XA-17 IgG (Full) | gD2 | — | IgG(Native) |
| 006 | XA-17 IgG (LFv) | gD2 | — | IgG(Fab*)-KiH |
| 007 | XA-17(K)/XV-17(H) IgG (LFv) | gD2 | gD2 | IgG(Fab*)-KiH |
| 008 | VEGFA(K)/XV-17(H) IgG (LFv) | VEGFA | gD2 | IgG(Native) |
| 009 | VEGFA IgG (Full) | VEGFA | — | IgG(Native) |
| 010 | VEGFA(K)/VEGFA (H) IgG (LFv) | VEGFA | VEGFA | IgG(Fab*)-KiH |
| 011 | XV-17(K)/VEGFA (H) IgG (LFv) | gD2 | VEGFA | IgG(Fab*)-KiH |
| 012 | Entyvio IgG(Full) | α4β7 | — | IgG(Native) |
| 013 | Entyvio(K)/Entyvio(H) IgG (LFv) | α4β7 | α4β7 | IgG(Fab*)-KiH |
| 014 | Humira(K)/Entyvio(H) IgG (LFv) | TNFα | α4β7 | IgG(Fab*)-KiH |
| 015 | Humira IgG(Full) | TNFα | — | IgG(Native) |
| 016 | Humira(K)/Humira(H) IgG (LFv) | TNFα | TNFα | IgG(Fab*)-KiH |
| 017 | Entyvio(K)/Humira(H) IgG (LFv) | α4β7 | TNFα | IgG(Fab*)-KiH |
| 018 | Avastin IgG(Full) | VEGFA | — | IgG(Native) |
| 019 | Avastin(K)/Avastin(H) IgG (LFv) | VEGFA | VEGFA | IgG(Fab*)-KiH |
| 020 | Herceptin(K)/Avastin(H) IgG (LFv) | Her2 | VEGFA | IgG(Fab*)-KiH |
| 021 | Herceptin IgG(Full) | Her2 | — | IgG(Native) |
| 022 | Herceptin(K)/Herceptin(H) IgG (LFv) | Her2 | Her2 | IgG(Fab*)-KiH |
| 023 | Avastin(K)/Herceptin(H) IgG (LFv) | VEGFA | Her2 | IgG(Fab*)-KiH |
| 024 | Herceptin IgG (LFv) | Her2 | — | IgG(Fab*) |
| 026 | Herceptin IgG (LFv)-Kds | Her2 | — | IgG(Fab*) |
| 028 | Entyvio(K)/Entyvio(H) IgG (LFv)-Kds | α4β7 | α4β7 | IgG(Fab*)-KiH-ds |
| 030 | Humira(K)/Entyvio(H) IgG (LFv)-Kds | TNFα | α4β7 | IgG(Fab*)-KiH-ds |
| 032 | Humira(K)/Humira(H) IgG (LFv)-Kds | TNFα | TNFα | IgG(Fab*)-KiH-ds |
| 034 | Entyvio(K)/Humira(H) IgG (LFv)-Kds | α4β7 | TNFα | IgG(Fab*)-KiH-ds |
| 036 | Entyvio(K)/Entyvio(H) BF-IgG | α4β7 | α4β7 | IgG(Fab**)-KiH |
| 037 | Humira(K)/Humira(H) BF-IgG | TNFα | TNFα | IgG(Fab**)-KiH |
| 038 | Humira(K)/Entyvio(H) BF-IgG | TNFα | α4β7 | IgG(Fab**)-KiH |
| 039 | Humira(K)/Humira(H) BF-IgG | α4β7 | TNFα | IgG(Fab**)-KiH |
| 042 | Entyvio(N)/Humira(C) Fab(LFv)-Kds-1G4S | α4β7 | TNFα | Fab*** |
| 043 | Humira(N)/Entyvio(C) Fab(LFv)-Kds-1G4S | TNFα | α4β7 | Fab*** |
| 053 | Avastin(K)/Herceptin(H) IgG(Full) | VEGFA | Her2 | IgG(Native)-KiH |

Note:
The abbreviation “K” refers to introduction of a “Knob” into CH3 domain of the respective heavy chain.
The abbreviation “H” refers to introduction of a “Hole” into CH3 domain of the respective heavy chain
The abbreviation “Kds” refers to the an presence of a disulfide bond in the kappa domain.
The abbreviation “KiH” refers to an antibody or bispecific antibody prepared using knobs-into holes strategy.
The abbreviation “ds” refers to “disulfide-bond stabilized” format.
The term “IgG(Native)” refers to an IgG antibody comprising native Fab.
The term “IgG(Fab*)” refers to an IgG antibody comprising Fab of the invention (1 linker).
The term “IgG(Fab**)” refers to an IgG antibody comprising Fab of the invention (2 linker).
The term “Fab***” refers (two Fabs connected with each other) comprising Fab of the invention (1 linker).

### Example 3: Sodium Dodecyl Sulfat-Polyacrylamide Gel Electrophoresis (SDS-PAGE)

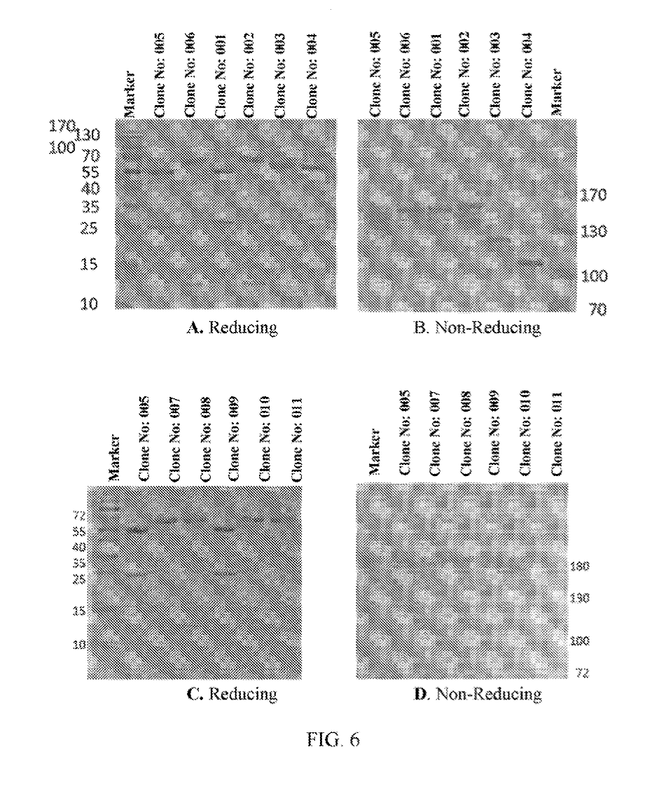

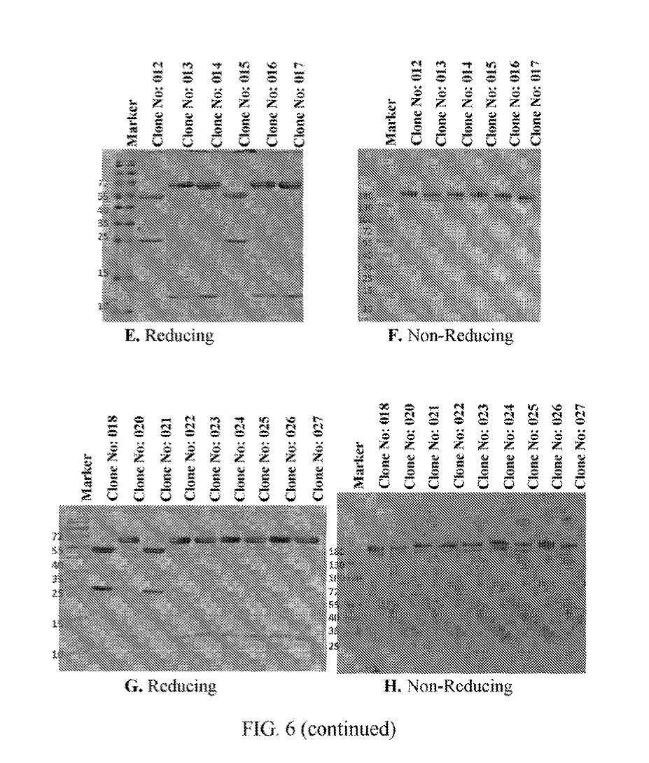

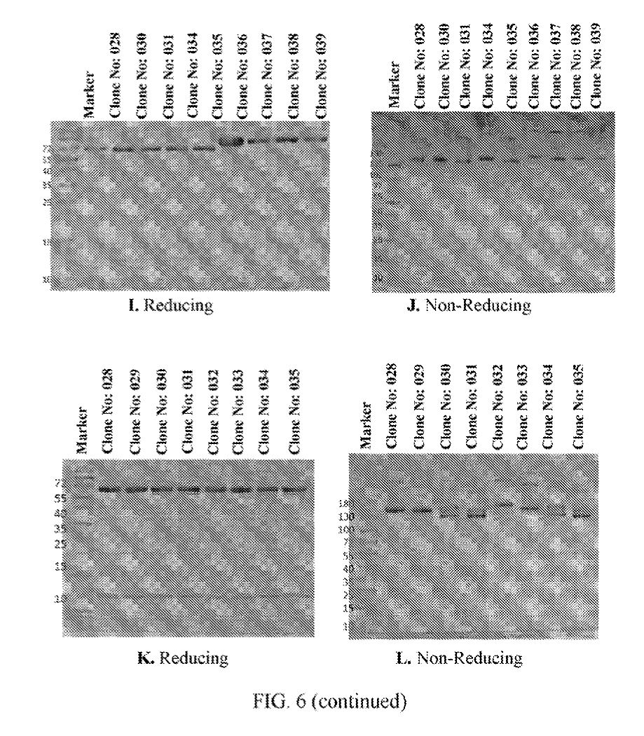

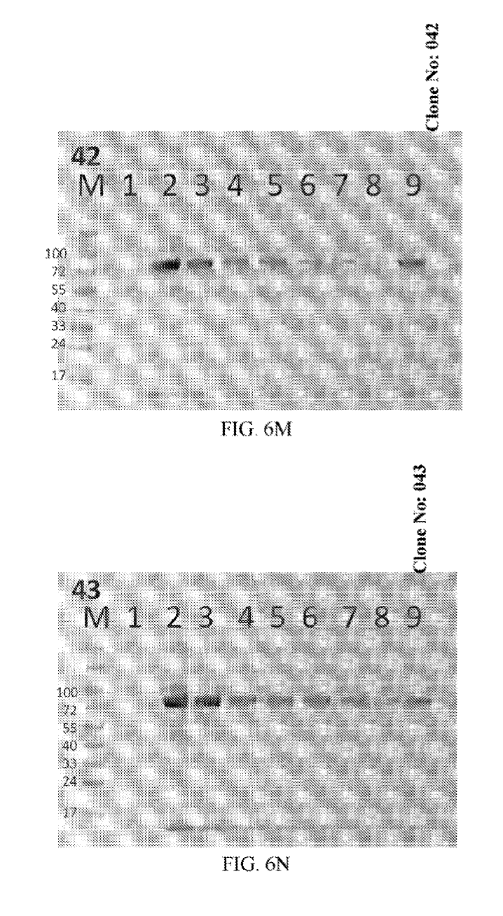

The clones obtained from Example 2 were analyzed by SDS-PAGE under reducing or non-reducing condition. FIG. 6A shows that the heavy and light chain fragments within Clone No: 005 construct have the expected size and distribution. A slight increase in size for the heavy chain fragment and decreased in size for the light chain fragment of Clone No: 006 construct can be observed. The slight change in size is as expected with Clone No: 006 and other constructs as well. FIG. 6B shows that, under anon-reducing condition, purified Clone No: 005 antibody has the molecular weight (MW) corresponding to that of the native form antibody. Clone No: 006 antibody on the other hand, shows a higher MW in the heavy chain region and a 1/2 lower MW in the light chain region when compared to the native antibody. Clone No: 001 and Clone No: 002 show similar banding patterns and the other constructs from Clone No: 007 to Clone No: 011 are also the same (FIGS. 6C and 5D). Under the non-reducing condition of SDS-page, all the antibodies show the same major MW band without dimers or aberrant aggregations. The same goes for other clones (FIGS. 6E to 6L). Clone No: 042 and Clone No: 043 were also analyzed by SDS-page, and the results are shown m FIGS. 6M and 6N, respectively. Lanes 1-8 in both FIGS. 6M and 6N refer to the SDS-page results of 8 fraction samples (obtained from the supernatants through protein L column) of Clone No: 042 and Clone No: 043, respectively. Lane 9 in both FIGS. 6M and 6N refers to the SDS-page result of concentrated samples of Clone No: 042 and Clone No: 043, respectively.

### Example 4: Capillary Electrophoresis (CE) Purity/Heterogeneity Assay

Materials for CE Purity/Heterogeneity assay include Capillary. 50 μm I.D. bare-fused silica, SDS-MW Gel buffer-proprietary formulation (pH 8, 0.2% SDS), SDS-MW Sample Buffer-100 mM Tris-HCl (pH 9.0, 1% SDS), IgG control standard, internal standard (10 kDa protein, 5 mg/mL), acidic wash solution (0.1 N HCl) and basic wash solution, 0.1 N NaOH.

Step 1: Preparation of the PA 800 Plus Instrument

Capillary replacement: install a 50 μm i.d. bare fused-silica capillary into a PA 800 plus cartridge set for a total capillary length of 30.2 cm. Installation of the PDA detector: turn on the instrument and permit the UV lamp to warm up for at least 30 minutes prior to experimentation.

Step 2: Preparation of Alkylation Reagent

250 mM iodoacetamide (IAM) solution was used as the alkylation reagent. The solution was stable for approximately 24 hours at room temperature. The preparation of IAM solution comprises the steps of weighing 46 mg of high purity IAM; transferring the IAM into a 1.5 mL centrifuge tube; adding 1 mL of DDI water to the 1.5 mL centrifuge tube; capping the vial tightly and mix thoroughly until dissolved; and making this mix fresh daily without and exposing it to the light.

Step 3: Preparation of the IgG Control Standard

The preparation of IgG control standard comprises the steps of taking 1 vial of the 95 μL aliquots of the IgG (1 mg/mL) control standard and setting it at room temperature until it is completely thawed; adding 2 μL of 10 kDa Internal Standard to the IgG tube, adding 5 μL of the 250 mM IAM to the IgG tube inside a fume hood; capping the tube and mixing thoroughly; centrifuging at 300 g for 1 minute; sealing the vial cap with parafilm and heating the mixture at 70° C. for 10 minutes; placing the vial in a room-temperature water bath to cool for at least 3 minutes; transferring 100 μL of the prepared sample into a micro vial; placing the micro vial into a universal vial; and capping the universal vial.

Step 4: Preparation of IgG Non-Reduced Sample

The preparation of IgG non-reduced sample comprises the steps of pipetting 100 μg of IgG sample into a 0.5 mL micro-centrifuge tube; adding from 50 to 95 μL of sample buffer to give a final volume of 95 μL; adding 2 μL of Internal Standard into the tube; adding 5 μL of the 250 mM IAM solution into the sample tube; capping the vial tightly and mixing thoroughly; centrifuging the sample tube at 300 g for 1 minute; sealing the sample tube with parafilm and heating the mixture in a water bath at 70′C for 10 minutes; placing the sample tube in a room temperature water bath to cool for at least 3 minutes; transferring 100 μL of the prepared sample into a 200 μL micro vial and spinning down the contents to remove any air bubbles; placing the micro vial inside a universal vial; and capping the universal vial.

### Example 5: BIAcore Assay

Surface plasmon resonance experiments were performed to evaluate the dual-binding behavior of the BsAbs using, for example, a Biacore T100 using HBS-EP+ as the running buffer (GE Healthcare). To determine the binding kinetics and affinity, an SPR-based assay on a BIAcore TI00 was performed. The binding kinetics was measured by single-cycle kinetics (SCK) method provided with the software. An anti-Human Ab was immobilized on CM5 chips at a density that would allow the maximum Response Units (RU) to be achieved. Choose Run: method, Select Assay/Kinetics/Affinity. Set the parameters as followed: Data collection rate 1 Hz. Detection mode Dual, Temperature 25° C., Concentration unit nM, Buffer A HBS-EP.

Select flow path 2-1, chip CM5, regeneration 1.

Select the Startup and change the Number of replicates to 3.

Select the Sample and set the parameters as followed, Contact time: 150 s, Flow rate: 40 μL/min, Dissociation time. 500 s.

Select the Regeneration and set the parameters as followed, Regeneration solution: 25 mM Glycine pH1.5, Contact time: 90 s, Flow Rate: 30 μL/min, Stabilization period: 90 s.

Serial dilutions of analyte with running buffer (HBS-EP+). The series concentration obtained as 40, 20, 10, 5, 2.5, 0. Prepare and position samples according to Rack Positions.

Evaluate result using BIAcore T100 evaluation software.

The binding responses were corrected for buffer effects by subtracting responses from a blank flow cell. A 1:1 Langmuir fitting model was used to estimate the kon (on-rate) and koff (off-rate). The KD values (the equilibrium dissociation constant between the antibody and its antigen) were determined from the ratios of kon and koff.

### Example 6: Assay Results of the Antigen-Binding Molecules of the Invention

The clones obtained from Example 2 were analyzed for their KD values on the target antigen according to BIAcore Assay of Example 3 as well as their recovery rate according to Mass spectrometry and CE of Example 4. The results are shown in Table 9 below.

TABLE 9

| | | | | | Recovery rate |
|---|---|---|---|---|---|
| Clone | Target 1 | | Target 2 | | (correctly |
| No. | target | KD | target | KD | folded rate) |
| 001 | HSA | 3.453E−10 | — | — | NA |
| 002 | HSA | 3.598E−10 | — | — | NA |
| 003 | HSA | 3.675E−10 | — | — | NA |
| 004 | HSA | 4.117E−10 | — | — | NA |
| 005 | gD2 | 4.769E−11 | — | — | 97% |
| 006 | gD2 | <E−11 | — | — | NA |
| 007 | gD2 | <E−11 | gD2 | <E−11 | 93% |
| 008 | VEGFA | 1.554E−9 | gD2 | <E−11 | NA |
| 009 | VEGFA | <E−11 | — | — | 96% |
| 010 | VEGFA | 3.661E−11 | VEGFA | 3.661E−11 | NA |
| 011 | gD2 | <E−11 | VEGFA | 1.553E−9 | 91% |
| 012 | α4β7 | 1 63E−10 | — | — | 97% |
| 013 | α4β7 | 2.53E−10 | α4β7 | 2.53E−10 | 87% |
| 014 | TNFα | 1.574E−10 | α4β7 | 9.65E−10 | 86% |
| 015 | TNFα | 6.430E−11 | — | — | 96% |
| 016 | TNFα | 1.633E−10 | TNFα | 1.633E−10 | 82% |
| 017 | α4β7 | 6.62E−10 | TNFα | 9.666E−11 | 80% |
| 018 | VEGFA | 3.940E−10 | — | — | 98% |
| 019 | VEGFA | 7.382E−12 | VEGFA | 7.382E−12 | 91% |
| 020 | Her2 | 1.586E−9 | VEGFA | 3.909E−8 | 86% |
| 021 | Her2 | 5.454E−10 | — | — | 100% |
| 022 | Her2 | 6.364E−10 | Her2 | 6.364E−10 | 86% |
| 023 | VEGFA | 8.204E−7 | Her2 | 7.340E−9 | 80% |
| 024 | Her2 | 5.703E−10 | — | — | 78% |
| 026 | Her2 | 6.150E−10 | — | — | 95% |
| 028 | α4β7 | 3.30E−10 | α4β7 | 3.30E−40 | 98% |
| 030 | TNFα | 1.540E−10 | α4β7 | 7.56E−10 | 97% |
| 032 | TNFα | 1.446E−10 | TNFα | 1.446E−10 | 96% |
| 034 | α4β7 | 6.82E−10 | TNFα | 2.056E−10 | 98% |
| 036 | α4β7 | 3.80E−10 | α4β7 | 3.80E−10 | 94% |
| 037 | TNFα | 1.813E−10 | TNFα | 1.813E−10 | 93% |
| 038 | TNFα | 1.723E−10 | α4β7 | 4.41E−10 | 91% |
| 039 | α4β7 | 5.49E−10 | TNFα | 1.631E−10 | 90% |
| 042 | α4β7 | 1.54E−10 | TNFα | 2.294E−8 | NA |
| 043 | TNFα | 7.051E−9 | α4β7 | 7.73E−10 | NA |
| 053 | VEGFA | NA | Her2 | NA | 0% |

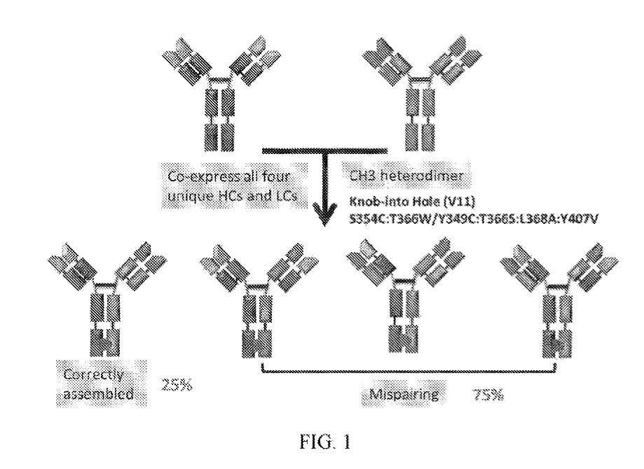

Clone No. 053 antibody refers to a BsAb having native IgG format and having KiH modification in CH3 domains. Theoretically. Clone No: 053 antibody is expected to have 25% recovery rate by using KiH strategy (see FIG. 1). Nevertheless, our result showed that no correctly assembled antibody was observed for Clone No: 053 clone. In comparison, Clone Nos: 013, 014, 016, 017, 019, 020, 022 and 023 refer to BsAbs comprising Fab of the invention and having KiH modification in CH3 domains and have more than 80% of recovery rate.

The BIAcore assay result showed no significant difference in the binding affinities of Clone No: 001 and Clone No: 002 and the same is observed with Clone No: 005 and Clone No: 006. For the bispecific antibody constructs. Clone No: 008 and Clone No 011 showed no differences in binding affinity to gD2, similar to Clone No: 005 and Clone No: 006. A 50-fold difference was observed in binding affinity for the VEGFA antigen between Clone No: 008/Clone No: 011 and Clone No: 009/Clone No: 010. The difference may be due to the antibody's transition from being bivalent to monovalent (Tables 8 and 9).

Clone Nos: 026, 028, 030, 032 and 034 were cysteine-engineered BsAbs, which were found to have a very high recovery rate (more than 95%). In addition, Clone No: 028 was found to have a higher stability than Clone No: 013 in nano-DSC thermal stability analysis.

These results showed that BsAb of the invention comprising novel Fab format can be robustly produced while retaining strong affinity to the antigens specifically.

Numerous modifications and variations of the invention as set forth in the above illustrative examples are expected to occur to those skilled in the art. Consequently, only such limitations as appearing in the appended claims should be placed on the invention.

1. An antigen-binding Fab fragment comprising a light chain variable domain (VL), a light chain constant domain (CL), a heavy chain variable domain (VH) and a heavy chain constant domain 1 (CH1), wherein the C-terminus of the VL domain is linked via a linker to the N-terminus of the VII domain; or the C-terminus of the VH domain is linked via a linker to the N-terminus of the VL domain, wherein:
(1) the VL domain is linked to the CL domain through a disulfide bond;
(2) the VH domain is linked to the CL domain through a disulfide bond;
(3) the VL domain is linked to the CH1 domain through a disulfide bond; or
(4) the VH domain is linked to the CH1 domain through a disulfide bond.

2. The antigen-binding Fab fragment of claim 1, wherein the C-terminus of the VL domain is linked via a linker to the N-terminus of the VH domain, and wherein:
(1) the C-terminus of the VH domain is linked to the N-terminus of the CH1 domain through a peptide bond;
(2) the C-terminus of the VH domain is linked to the N-terminus of the CL domain through a peptide bond;
(3) the C-terminus of the VH domain is linked to the N-terminus of the CL domain through a peptide bond, and the C-terminus of the CL domain is linked via a linker to the N-terminus of the CH1 domain; or
(4) the C-terminus of the VH domain is linked to the N-terminus of the CH1 domain through a peptide bond, and the C-terminus of the CH1 domain is linked via a linker to the N-terminus of the CL domain.

3. The antigen-binding Fab fragment of claim 1, wherein the C-terminus of the VH domain is linked via a linker to the N-terminus of the VL domain, and wherein:
(1) the C-terminus of the VL domain is linked to the N-terminus of the CH1 domain through a peptide bond;
(2) the C-terminus of the VL domain is linked to the N-terminus of the CL domain through a peptide bond;
(3) the C-terminus of the VL domain is linked to the N-terminus of the CL domain through a peptide bond, and the C-terminus of the CL domain is linked via a linker to the N-terminus of the CH1 domain; or
(4) the C-terminus of the VL domain is linked to the N-terminus of the CH1 domain through a peptide bond, and the C-terminus of the CH1 domain is linked via a linker to the N-terminus of the CL domain.

4. (canceled)

5. The antigen-binding Fab fragment of claim 1, further comprising an Fc region.

6. The antigen-binding Fab fragment of claim 2, further comprising an Fc region.

7. The antigen-binding Fab fragment of claim 3, further comprising an Fc region.

8. (canceled)

9. An antigen-binding molecule comprising two or more of the antigen-binding Fab fragments of claim 1, wherein the two or more of the fragments are specific to identical or different antigens.

10. An antigen-binding molecule comprising two or more of the antigen-binding Fab fragments of claim 2, wherein the two or more of the fragments are specific to identical or different antigens.

11. An antigen-binding molecule comprising two or more of the antigen-binding Fab fragments of claim 3, wherein the two or more of the fragments are specific to identical or different antigens.

12. An antigen-binding molecule comprising two or more of the antigen-binding Fab fragments of claim 4, wherein the two or more of the fragments are specific to identical or different antigens.

13. An antigen-binding molecule comprising two or more of the antigen-binding Fab fragments of claim 5, wherein the two or more of the fragments are specific to identical or different antigens, and wherein the antigen-binding molecule has an Fc region.

14. An antigen-binding molecule comprising two or more of the antigen-binding Fab fragments of claim 6, wherein the two or more of the fragments are specific to identical or different antigens, and wherein the antigen-binding molecule has an Fc region.

15. (canceled)

16. (canceled)

17. A polynucleotide encoding the antigen-binding Fab fragment of claim 1.

18. A polynucleotide encoding the antigen-binding Fab fragment of claim 2.

19. A polynucleotide encoding the antigen-binding Fab fragment of claim 3.

20. A polynucleotide encoding the antigen-binding Fab fragment of claim 4.

21. A polynucleotide encoding the antigen-binding Fab fragment of claim 5.

22. A polynucleotide encoding the antigen-binding Fab fragment of claim 6.

23. (canceled)

24. (canceled)

25. A vector comprising one or more polynucleotides encoding one or more of the antigen-binding Fab fragments of claim 1.

26. A vector comprising one or more polynucleotides encoding one or more of the antigen-binding Fab fragments of claim 2.

27. A vector comprising one or more polynucleotides encoding one or more of the antigen-binding Fab fragments of claim 3.

28. A vector comprising one or more polynucleotides encoding one or more of the antigen-binding Fab fragments of claim 4.

29. A vector comprising one or more polynucleotides encoding one or more of the antigen-binding Fab fragments of claim 5.

30. A vector comprising one or more polynucleotides encoding one or more of the antigen-binding Fab fragments of claim 6.

31. (canceled)

32. (canceled)

33. A host cell comprising the vector of claim 25.

34. A host cell comprising the vector of claim 26.

35. A host cell comprising the vector of claim 27.

36. A host cell comprising the vector of claim 22.

37. A host cell comprising the vector of claim 29.

38. A host cell comprising the vector of claim 30.

39. (canceled)

40. (canceled)

41. A method of preparing an antigen-binding molecule, which comprises incubating the host cell of claim 33.

42. A method of preparing an antigen-binding molecule, which comprises incubating the host cell of claim 34.

43. A method of preparing an antigen-binding molecule, which comprises incubating the host cell of claim 35.

44. A method of preparing as antigen-binding molecule, which comprises incubating the host cell of claim 36.

45. A method of preparing an antigen-binding molecule, which comprises incubating the host cell of claim 37.

46. A method of preparing an antigen-binding molecule, which comprises incubating the host cell of claim 38.

47. (canceled)

48. (canceled)

49. A pharmaceutical composition comprising the antigen-binding molecule of claim 9 and a pharmaceutically acceptable carrier.

50. A pharmaceutical composition comprising the antigen-binding molecule of claim 10 and a pharmaceutically acceptable carrier.

51. A pharmaceutical composition comprising the antigen-binding molecule of claim 11 and a pharmaceutically acceptable carrier.

52. A pharmaceutical composition comprising the antigen-binding molecule of claim 12 and a pharmaceutically acceptable carrier.

53. A pharmaceutical composition comprising the antigen-binding molecule of claim 13 and a pharmaceutically acceptable carrier.

54. A pharmaceutical composition comprising the antigen-binding molecule of claim 14 and a pharmaceutically acceptable carrier.

55. (canceled)

56. (canceled)
